(19) United States
(12) Patent Application Publication
(10) Pub. No.: US 2016/0257988 A1
(43) Pub. Date: Sep. 8, 2016

(54) Indicator Molecules For Use In Detecting Enzyme Cleavage Activity

(71) Applicant: Mologic Limited, Thurleigh, Bedfordshire (GB)

(72) Inventors: Paul Davis, Sharnbrook, Bedfordshire (GB); Gita Parekh, Milton Keynes, Bedfordshire (GB)

(73) Assignee: Mologic Limited, Thurleigh, Bedfordshire (GB)

(21) Appl. No.: 15/031,608

(22) PCT Filed: Oct. 23, 2014
(86) PCT No.: PCT/GB2014/053166

§ 371 Date: Apr. 22, 2016

(30) Foreign Application Priority Data
Oct. 23, 2013 (GB) 1318729.9

(51) Int. Cl. C12Q 1/37; G01N 21/64; G01N 21/77

(52) U.S. Cl. CPC C12Q 1/37; G01N 21/77; G01N 21/6428; G01N 2021/6432

(57) ABSTRACT

The present invention relates to indicator molecules containing multiple cleavage sites for use in detecting enzyme cleavage activity. The invention also relates to various applications of these indicator molecules, for example in enzyme detection devices, particularly although not exclusively, devices for use in the detection of enzyme activity in a test sample. The invention also relates to using the indicator molecules in methods for detecting the presence of enzyme activity in a test sample.

### FIELD OF THE INVENTION

The present invention relates to indicator molecules containing multiple cleavage sites for use in detecting enzyme cleavage activity. The invention also relates to various applications of these indicator molecules, for example in enzyme detection devices, particularly although not exclusively, devices for use in the detection of enzyme activity in a test sample. The invention also relates to using the indicator molecules in methods for detecting the presence of enzyme activity in a test sample.

### BACKGROUND TO THE INVENTION

Enzymes constitute a family of proteins involved in catalysing chemical reactions. As a result of their importance, there are numerous situations in which it is necessary and/or beneficial to measure enzyme levels, and importantly, enzyme activity.

In particular, changes in enzyme activity have been found to correlate with specific conditions and/or diseases. For example up-regulated protease activity has been associated with many aspects of cancer progression. The measurement of enzyme activity in samples taken from individuals with a particular condition or suspected of having a specific condition or disease may therefore be useful for prognostic or diagnostic purposes.

Within the enzyme family, there are many classes of enzyme that act by facilitating substrate cleavage. For example, peptidases and proteases catalyse the hydrolysis of peptide bonds within their respective substrates. In the past, researchers have, in some cases, sought to measure this type of activity using kits or devices that measure release of a fragment or ‘leaving group’ from the initial enzyme substrate.

Assays based on this fundamental principle have been refined such that in some cases, inventors have described engineered substrate molecules linked to reporter moieties. Cleavage of the substrate by the enzyme to be detected, if present, leads to release of said reporter, which can be detected by a range of techniques available to those skilled in the art. An assay of this type is described for example in US2006/0003394.

Others have sought to develop assays for the measurement of enzyme activity based around the principle of discriminating between modified and unmodified forms of an enzyme substrate. In this regard, WO2009/024805 describes an enzyme detection device utilising a “substrate recognition molecule” (SRM) carrying a detectable label, wherein the SRM specifically binds to the enzyme substrate in either the unmodified or modified state.

### SUMMARY OF INVENTION

The present invention seeks to improve sensitivity of detection of enzyme cleavage activity. The inventors have found that improved sensitivity of detection of enzyme cleavage activity can be achieved by incorporating multiple separate cleavage sites into the indicator molecules acted upon by the enzyme. The indicator molecules comprise a flurophore such that cleavage produces a measurable change in fluorescence of the indicator molecule.

Accordingly, the present invention provides an indicator molecule for use in the detection of enzyme cleavage activity in a test sample comprising:
(i) an enzyme modifiable region comprising multiple separate cleavage sites; and
(ii) a fluorophore wherein cleavage at one or more of the multiple separate cleavage sites causes a measurable change in fluorescence of the fluorophore.

The fluorophore acts as a label to enable detection of cleavage at one or more of the multiple separate cleavage sites. In specific embodiments, the measurable change in fluorescence is intensity, polarization or lifetime. Fluoroscence polarization measurements may be advantageous in some embodiments because they may be less susceptible to environmental interference, for example caused by pH changes, than corresponding fluorescence intensity measurements. Where fluorescence polarization measurements are made, the indicator molecule may be labelled with a single fluorophore. Suitable fluorophores include Cy3B (GE Healthcare) and BODIPY (Life Technologies) dyes.

In particular embodiments, cleavage of the indicator molecule produces a measurable change in fluorescence due to a change in fluorescence resonance energy transfer (FRET). Thus, in some embodiments the indicator molecule further comprises a second fluorophore to create a donor and acceptor transfer pair. In such embodiments, the measurable change in fluorescence intensity is caused by the separation of the donor and acceptor transfer pair upon cleavage at any one of the multiple separate cleavage sites. Typically, cleavage results in a decrease in fluorescence emission by the acceptor and a corresponding increase in fluorescence emission by the donor. In certain embodiments, the indicator molecule comprises the structure FLUORESCENCE DONOR-[CLEAVAGE SITE]n-FLUORESCENCE ACCEPTOR, wherein n is at least 2. The donor and acceptor fluorophores flank the multiple separate cleavage sites. The donor and/or acceptor fluorophores may be separated from the cleavage sites by spacer or linker regions. Thus in the structure above “-” may comprise a spacer or linker region to separate the cleavage sites from the fluorophores in some embodiments. The number of separate cleavage sites that may be included is discussed in detail herein, but may be up to 25 in some embodiments.

In particular embodiments, the acceptor is a quencher. Here, instead of the transferred energy being re-emitted at a longer wavelength by the acceptor it is converted to non-fluorescence energy. In some embodiments the fluorescence intensity of the donor is increased upon cleavage at any one of the multiple separate cleavage sites. There are a range of donor-acceptor pairs that may be incorporated into the indicator molecules of the invention as would be readily understood by one skilled in the art. Many are commercially available. Typically, in order to maximise the FRET effect, the absorbance spectrum of the acceptor/quencher and the emission spectrum of the fluorophore should have similar maxima. Suitable molecules can be selected based upon the preferred emission wavelength. An example of an acceptor is QSY35 (Life Technologies) which absorbs at 475 nm and can form a FRET pair with any of chromis 425N (Cyanagen), Mca, AF405, AMCA-X, AF350, EDANS, chromis 500N, bodipy FL-X, OG488, AF488 and FAM. Another example is Dabcyl which absorbs at a wavelength of 453 nm and can form a FRET pair with any of chromis 425N (Cyanagen), AF405, AMCA-X, AF350 and EDANS. At a longer wavelength, for example Alexa Fluor610 (Life Technologies) absorbs at a wavelength of around 604 nm and emits at 623 nm. This fluorophore can form a FRET pair with a range of molecules including QSY21, BHQ2, BHQ3 and IRDye QC-1. Other commonly used FRET pairs include fluorescein and dabcyl, fluorescein and tamra; and methoxy-coumarin acetic acid and 2,4-dinitrophenyl.

Typically, enzyme substrates based on FRET have been designed to contain the minimum recognition sequence for a protease, with a donor and acceptor/quencher fluorophore attached at either end. This is because the efficiency of FRET is inversely proportional to the sixth power of the distance between the donor and acceptor/quencher fluorophore. Thus, prior to the invention, substrates have incorporated a single cleavage site to ensure that the donor and acceptor/quencher remain in sufficient proximity prior to cleavage to achieve efficient FRET. The inventors have found that, in stark contrast to this approach, incorporating multiple separate cleavage sites into the indicator molecules of the invention (and separating the donor and acceptor fluorophores) can actually improve sensitivity of detection of enzyme cleavage activity.

The indicator molecule of the invention may generally comprise multiple cleavage sites wherein cleavage at any one of the cleavage sites results in a measurable change in fluorescence. Accordingly, in some embodiments, the indicator molecule comprises between 2 and 25 separate cleavage sites. Thus, the indicator molecule may comprise 2, 3, 4, 5, 6, 7, 8, 9, 10, 11, 12, 13, 14, 15, 16, 17, 18, 19, 20, 21, 22, 23, 24 or 25 cleavage sites.

In certain embodiments, the indicator molecule includes between 2, 3, 4, 5 and 10, 11, 12, 13, 14, 15, 16, 17, 18, 19, 20, 25, 50, 100, 500 or 1000 separate cleavage sites. Higher numbers of cleavage sites may be more applicable to indicator molecules used in non-FRET based detection techniques such as fluorescence polarization changes associated with a fluorophore as a consequences of enzyme cleavage. In some embodiments, the indicator molecule includes between 2 and 5, 6, 7, 8, 9 or 10 cleavage sites.

The multiple separate cleavage sites define an enzyme modifiable region of the indicator molecule. The fluorophore is generally bound to the indicator molecule outside of the enzyme modifiable region. Attachment of the fluorophore is generally direct, for example by covalent linkage, but may be indirect in some embodiments.

The multiple cleavage sites may be separated from one another by virtue of linker or spacer regions. At a minimum, the site of cleavage will be presented in a suitable context, for example, flanked by suitable amino acids, to ensure cleavage occurs as efficiently and/or specifically as required. Thus, by “cleavage site” is meant a region comprising the minimum recognition sequence for the cleavage enzyme or enzymes to be detected. However, the linkers or spacers may be used to further separate the cleavage sites from one another. This may enhance access to the cleavage sites by the enzyme or enzymes and thus improve sensitivity. The linkers or spacers may separate the multiple cleavage sites from the fluorophore(s) and/or may be found between one or more cleavage sites within the indicator molecule.

In some embodiments, the multiple cleavage sites may all be identical. In this configuration, the repeated cleavage site may be relatively non-specific or may be highly specific for one enzyme or enzyme subtype. Use of an indicator molecule of this type may help to increase the sensitivity of enzyme detection by providing a means to increase the concentration of cleavage sites present within the test sample.

In other embodiments, the indicator molecule may comprise multiple cleavage sites wherein there are at least two different cleavage sites present within the same indicator molecule. In preferred embodiments of the invention, the indicator molecule may comprise at least three, at least four, at least five, and up to at least 8 different cleavage sites. In further embodiments, the different cleavage sites are recognised by different enzymes or different categories, subcategories or subtypes of enzymes, such that the device of the invention can be used to detect the activity of multiple different enzymes. The activities may be grouped, such that the detection of enzyme activity gives a useful result. For example, a group of enzymes may be involved in a disease state such that detection of the relevant activity of one or more of the enzyme group is diagnostically useful.

Use of multiple cleavage sites (whether identical or non-identical) may be particularly useful for situations in which very low levels of enzyme activity are to be detected in a test sample. For example, an indicator molecule having multiple cleavage sites as defined above may be used to detect enzyme activity in a urine sample containing low levels of protease.

The enzyme or enzymes to be detected using the indicator molecules described herein must be capable of cleaving substrates. This activity is required in order for the indicator molecule to be split into at least two separate fragments, causing a measurable change in fluorescence of the at least one fluorophore. Typically the enzyme or enzymes to be detected are proteases, which term is defined herein to explicitly include peptidases. Protease subtypes that may be detected include: Serine proteases, threonine proteases, cysteine proteases, aspartate proteases, glutamic-acid proteases and metalloproteases. Specific examples of enzymes that may be detected using the indicator molecules of the invention include proteases, and in particular matrix metalloproteases (MMPs) and human neutrophil-derived elastase (HNE). In some embodiments, the enzyme to be detected is a cathepsin, in particular cathepsin G.

In certain embodiments of the invention, at least one of the multiple separate cleavage sites may be relatively non-specific such that at least one of the cleavage sites is capable of being recognised or acted on by more than one enzyme. For example, a simple peptide substrate may be subject to cleavage by a number of different proteases. In embodiments wherein the cleavage sites are recognised or acted on by more than one enzyme, the indicator molecules of the invention may be used to detect the presence in a test sample of any of such enzymes capable of cleaving the indicator molecule.

In other embodiments of the invention, the at least one of the multiple separate cleavage sites of the indicator molecule may be highly specific such that only one enzyme or one sub-type of enzyme is capable of recognising and cleaving the cleavage site. For example, at least one of the multiple separate cleavage sites may be recognised by a single protease or sub-type of proteases. Protease subtypes may include the following: serine proteases; threonine proteases; cysteine proteases; aspartate proteases; metalloproteases and glutamic acid proteases. In such embodiments, the indicator molecule may be tailored to the detection of a single enzyme or single enzyme subtype within a test sample.

In preferred embodiments, the multiple cleavage sites are preferentially cleaved by one or more specific proteases. In specific embodiments, the multiple cleavage sites are preferentially cleaved by one or more matrix metalloproteases. In further embodiments, at least one (up to all) of the multiple cleavage sites is preferentially cleaved by MMP-13 and/or MMP-9. In other embodiments, at least one (up to all) of the multiple cleavage sites is preferentially cleaved by MMP-13, 9, 2, 12 and 8, optionally in that order of preference, compared to other MMPs. In other embodiments, one or more cleavage sites are preferentially cleaved by a particular protease and one or more further cleavage sites are preferentially cleaved by a different protease. Specifically, the indicator molecules of the invention may be used to detect the presence or absence of any one of at least two, three, four or five different proteases.

As already mentioned, the at least one cleavage site may be biased for cleavage by MMP-13, 9, 2, 12 and 8. The bias may be for the group of MMPs equally or may be in that particular order of preference. It is possible to design specific indicator molecules and cleavage sites within the indicator molecules that are biased for cleavage by these particular MMPs, in the specified order of preference. Accordingly, in some embodiments at least one, up to all, of the multiple cleavage sites are found within the amino acid sequence GPQGIFGQ (SEQ ID NO: 1). In those embodiments, cleavage of the peptide bond between the glycine and isoleucine residue produces a part of the indicator molecule containing the amino acid sequence GPQG and a part of the indicator molecule containing the amino acid sequence IFQG. Thus the multiple cleavage sites may comprise repeats of SEQ ID NO: 1. The repeats may be separated by a spacer or linker as described herein.

The indicator molecules disclosed herein may be useful in a variety of assay formats. They may be useful in homogenous, or solution phase, assays. Thus, cleavage results in a measurable change in fluorescence which can be measured in solution without any requirement for further separation of the components. In some embodiments, the indicator molecule does not contain a separate capture site which can be bound by a capture molecule irrespective of the state of modification of the enzyme modifiable region.

The indicator molecules may advantageously be immobilised when used in various detection methodologies. Thus, in some embodiments, the indicator molecule contains a capture site (which wording is intended to encompass at least one capture site). In some embodiments, the capture site can be bound by a capture molecule irrespective of the state of modification of the enzyme modifiable region. The interaction between capture site and capture molecules can thus be used to immobilize the indicator molecule on a solid support. The capture molecules may form a defined capture zone on the solid support in some embodiments. Any suitable solid support may be used (as discussed further herein).

The capture site is a discrete region of the indicator molecule which permits immobilization of the indicator molecule, generally whether cleaved or uncleaved, on a solid support. Thus, the capture site is the portion of the indicator molecule responsible for retaining or localising the indicator molecule on a solid support, optionally within a defined capture zone. Following cleavage of the indicator molecule, the capture site may remain intact or substantially intact, such that the site is still recognised and bound by a capture molecule present on the solid support. Under these circumstances, both intact indicator molecules and the part or fragment of the indicator molecules comprising the capture site following cleavage will be bound to capture molecules. The capture site may comprise any suitable molecule, for example a biotin molecule.

As noted, the cleavage site may be within a peptide or a protein. In specific embodiments of the invention, the multiple separate cleavage sites and capture site are defined by discrete amino acids or groups of amino acids within a peptide or protein. As used herein the term “peptide” is intended to mean a length of amino acids of no more than (about) 20, 30, 40 or 50 amino acids. Alternatively, the capture site may be present in a region of the indicator molecule which is separate to the enzyme modifiable region in which the multiple cleavage sites are located. Thus, in certain embodiments of the invention, the capture site may be present within a capture region, and the multiple cleavage sites are present within a separate enzyme modifiable, or “cleavage”, region of the indicator molecule. In embodiments wherein the capture site is in a separate region of the indicator molecule to the cleavage region, the capture site may comprise materials or residues entirely distinct from those found in the cleavage region of the molecule containing the multiple cleavage sites. For example, the cleavage region may comprise amino acid residues whilst the capture site may comprise or consist of a biotin moiety. Moreover, in embodiments wherein the indicator molecule comprises separate regions bearing the multiple cleavage sites and capture site, said regions may be associated by any means known to one of skill in the art. In a preferred embodiment, said regions may be associated via a direct covalent linkage. Said regions may be immediately adjacent or may be separated by a linker or spacer, for example, a polyethylene glycol moiety.

Within the context of the present invention the indicator molecules (via the capture site) may bind to the capture molecules with relatively high affinity. In some embodiments, the dissociation constant (kd) for the indicator molecule will be relatively low and preferably between 0M and 1×10⁻⁷M (depending on the sensitivity required of the assay). In certain embodiments of the invention, the dissociation constant for the indicator molecule will be between 1×10⁻¹⁵M and 1×10⁻⁹M.

In certain embodiments of the invention, such a binding interaction may be achieved as a result of direct binding of the capture site of the indicator molecule to the capture molecule present in the capture zone. In this context, direct binding means binding of the indicator molecule (via the capture site) to the capture molecule without any intermediary.

In some embodiments of the invention, the capture site of the indicator molecule and the capture molecule present in the capture zone are two halves of a binding pair. In this context, a binding pair consists of two molecules or entities capable of binding to each other. In certain embodiments of the invention, the binding interaction is specific such that each member of the binding pair is only able to bind its respective partner, or a limited number of binding partners. Moreover, as detailed above, it is preferable for the binding pair to exhibit relatively high affinity. The binding pair may be a binding pair found in nature or an artificially generated pair of interacting molecules or entities.

In some embodiments of the invention, the capture site of the indicator molecule and the capture molecule are two halves of a binding pair wherein the binding pair is selected from the following:—an antigen and an antibody or antigen binding fragment thereof; biotin and avidin, streptavidin, neutravidin or captavidin; an immunoglobulin (or appropriate domain thereof) and protein A or G; a carbohydrate and a lectin; complementary nucleotide sequences; a ligand and a receptor molecule; a hormone and hormone binding protein; an enzyme cofactor and an enzyme; an enzyme inhibitor and an enzyme; a cellulose binding domain and cellulose fibres; immobilised aminophenyl boronic acid and cis-diol bearing molecules; and xyloglucan and cellulose fibres and analogues, derivatives and fragments thereof.

In particular embodiments of the invention, the binding pair consists of biotin and streptavidin. In a further embodiment of the invention, the capture site of the indicator molecule comprises an epitope and the capture molecule comprises an antibody, which specifically binds to the epitope present at the first capture site. In the context of the present invention, the term antibody covers native immunoglobulins from any species, chimeric antibodies, humanised antibodies, F(ab′)2 fragments, Fab fragments, Fv fragments, sFv fragments and highly related molecules such as those based upon antibody domains which retain specific binding affinity (for example, single domain antibodies). The antibodies may be monoclonal or polyclonal. Thus, in specific embodiments, the capture molecule comprises an antibody. In other embodiments, the capture site comprises a biotin molecule and the capture zone comprises a streptavidin molecule.

In certain embodiments of the invention, binding of the capture site of the indicator molecule to the capture molecule of the device may be indirect. In the context of the present invention, “indirect binding” means binding mediated by some intermediate entity capable of bridging the capture site of the indicator molecule and the capture molecule, for example an “adaptor” capable of simultaneously binding the capture site of the indicator molecule and the capture molecule.

Wherein binding of the indicator molecule to the capture molecule is indirect and mediated by an adaptor, it may be possible for a plurality of indicator molecules to bind to each capture molecule. In this context, a plurality means at least two, at least three, at least four, and so forth. This may be achieved by the incorporation of a multivalent adaptor molecule, for example, a streptavidin molecule capable of simultaneous binding to multiple biotin-containing indicator molecules in addition to a capture molecule consisting of or comprising biotin.

In a related aspect, the invention also provides for use of an indicator molecule as described and defined herein for detecting enzyme cleavage activity in a test sample. Similarly, the invention provides a method of detecting enzyme cleavage activity in a test sample, the method comprising:
a. bringing an indicator molecule into contact with the test sample, said indicator molecule comprising
   i. an enzyme modifiable region comprising multiple separate cleavage sites; and
   ii. a fluorophore,
wherein cleavage at one or more of the multiple separate cleavage sites causes a measurable change in fluorescence of the fluorophore.
b. detecting cleavage of at least one of the multiple separate cleavage sites by measuring a change in fluorescence of the fluorophore.

The indicator molecule can be an indicator molecule as defined anywhere within the specification. The indicator molecules may be used to test any suitable sample. In all aspects of the invention, the test sample may be any material known or suspected to contain an enzyme with cleavage activity. The test sample may be derived from any source. In certain embodiments, the test sample may be derived from a biological source including fluids such as blood (including serum and plasma), saliva, urine, milk, fluid from a wound, ascites fluid, peritoneal fluid, amniotic fluid and so forth. In some embodiments, the test sample is wound fluid and the indicator molecule is used to detect enzyme activity, preferably protease activity, in the wound fluid as a means to assess the status and/or rate of healing of a wound. In specific embodiments, the test sample is urine and the device is used to detect the activity of enzymes, in particular proteases, in the urine. In other embodiments, the sample may be an environmental sample in which cleavage activity may be desirably tested. For example, the sample may be a water, food or dust sample. In the context of food and drink samples, cleavage activity may be detected for example in relation to shelf-life of the product. Samples may also be laboratory or industrial samples, for example to test for proteases or other cleavage enzymes as contaminants. The contaminants may be found during various laboratory processes such as protein purification or in industrial processes such as fermentations.

The test sample may be collected by any suitable means and presented in any form suitable for use with the present invention, including solid or liquid forms. Moreover, as part of obtaining the test sample from its original source, the sample may undergo one or more processing or pre-treatment steps prior to testing using the invention. In one embodiment, a solid sample may be processed so as to produce a solution or suspension for testing. Moreover, in certain embodiments, the test sample may be stored, for example frozen at a suitable temperature (e.g. around −20° C.), as a means of preserving the sample for any given length of time prior to testing using the invention.

It should be noted that the invention is typically performed in vitro based upon isolated samples. The methods of the invention may include steps of obtaining a sample for testing in some embodiments.

In some embodiments, the use or method are performed in solution. In some embodiments the use or method are homogenous assays. In specific embodiments, none of the components are immobilised in performing the methods. In other embodiments, the indicator molecule is immobilized on a solid support. In specific embodiments cleavage at one or more of the multiple separate cleavage sites releases the fluorophore into solution such that in step b. of the method cleavage is detected in solution. In specific embodiments, the solid support is removed prior to detecting cleavage in solution. In further embodiments, cleavage of the indicator molecule releases the fluorophore. The released flurophore passes through a membrane permeable to the fluorophore, to allow detection in a separate detection zone. In such embodiments, the membrane is impermeable to the solid support to ensure that the solid support carrying the fluorophore does not pass into the separate detection zone (i.e. uncleared indicator molecules are not detected). The membrane is thus a semi-permeable or selectively-permeable membrane. The membrane may have a size or molecular weight cut-off. Suitable membranes and materials for construction of these membranes are well known to those skilled in the art and commercially available. It should be noted that the released fluorophore may remain attached to a portion, part or fragment of the indicator molecule, which may include one or more cleavage sites depending upon where cleavage occurred.

The invention also relates to a corresponding enzyme detection device for detecting enzyme cleavage activity in a test sample comprising:
an indicator molecule of the invention; and
a housing comprising:
   i. a reaction zone to which the indicator molecule and test sample is added
   ii. a detection zone where fluorescence of the fluorophore is detected; and
   iii. a membrane separating the reaction zone and detection zone, wherein the membrane is permeable to the fluorophore, or fragment of the indicator molecule containing the fluorophore, which is produced upon cleavage of the indicator molecule, but which is not permeable to the indicator molecule prior to cleavage.

In some embodiments, the enzyme detection device further comprises a solid support upon which the indicator molecule may be, or is, immobilized. In specific embodiments, the indicator molecule further comprises a second fluorophore to create a donor and acceptor transfer pair. Here, the membrane is permeable to the donor fluorophore, or fragment of the indicator molecule containing the donor fluorophore which is produced upon cleavage of the indicator molecule. In some embodiments the membrane is not permeable to the indicator molecule prior to cleavage or the fragment of the indicator molecule containing the acceptor fluorophore which is produced upon cleavage of the indicator molecule.

The invention also relates to the use of this enzyme detection device for detecting enzyme cleavage activity in a sample. Similarly, the invention further relates to a method of detecting enzyme cleavage activity in a test sample, the method comprising applying the test sample to a reaction zone of the enzyme detection device and detecting cleavage of one or more of the multiple separate cleavage sites by measuring fluorescence of the fluorophore in the detection zone.

The invention also provides an enzyme detection device for detecting the presence in a test sample of cleavage activity of an enzyme capable of cleaving a substrate, the device comprising:
a. an indicator molecule of the invention (as defined herein), wherein the indicator molecule contains a separate capture site which can be bound by a capture molecule irrespective of the state of modification of the enzyme modifiable region
b. a solid support comprising a capture zone to receive the test sample, wherein the capture zone comprises capture molecules capable of binding to the capture site of the indicator molecule.

This device allows multiple different assays to be performed based upon immobilization of the indicator molecule on the solid support, as discussed herein.

The capture zone is defined in further detail herein. In specific embodiments, following cleavage, the fluorophore remains immobilized at the capture zone by virtue of its association with the separate capture site. Thus, cleavage does not release the fluorophore from the capture zone in some embodiments.

The device may, in some embodiments, contain a detection zone. This detection zone may incorporate binding molecules which bind to the released fragment of the indicator molecule following cleavage. This acts to immobilise the released fluorophore, typically the donor in the embodiments where a donor and acceptor pair are employed, (although this orientation can be reversed in some embodiments) in a specific location where the label can then be detected. The detection zone may be formed on the same or a different solid support to the capture zone depending upon the nature of the test. The detection zone is spatially separated from the capture zone.

The binding molecules provide a specific binding interaction with the product of cleavage of the indicator molecule. Thus, the binding molecules locate the cleavage products, which contain a fluorophore to permit detection, in a detection region. Because the cleavage sites are known, the binding molecules can be rationally designed to bind to the products of cleavage containing the fluorophore. Typically, the binding molecule comprises an antibody. An antibody can be produced according to techniques well known to the skilled person in order to specifically bind to the products of cleavage that include the fluorophore. In the context of the present invention, the term antibody covers native immunoglobulins from any species, chimeric antibodies, humanised antibodies, F(ab′)2 fragments, Fab fragments, Fv fragments, sFv fragments and highly related molecules such as those based upon antibody domains which retain specific binding affinity (for example, single domain antibodies). The antibodies may be monoclonal or polyclonal.

Because the indicator molecules contain multiple cleavage sites, there are potentially a number of different cleavage products containing the fluorophore which could be formed, containing no, or one or more, intact cleavage sites connected to the fluorophore. It is possible to generate binding molecules that will bind to the same consensus sequence, which may be for example a spacer or linker after the final cleavage site that links the cleavage region to the fluorophore. Alternatively, multiple binding molecules (e.g. polyclonal antibodies) may be utilised that would bind to the various possible cleavage products incorporating the fluorophore.

The binding molecules may be directly or indirectly immobilized on or in the detection zone (solid support). In the context of the present invention, “indirect immobilization” means immobilization mediated by some intermediate entity, referred to as an “adaptor” capable of itself being directly immobilized on the solid support and also of binding to the binding molecules. Thus, the binding molecule may carry a biotin label. There may then be streptavidin molecules immobilized on the solid support to indirectly immobilize the binding molecules within the detection zone. In such embodiments, it may be possible for a plurality of binding molecules to bind to each adaptor. In this context, a plurality means at least two, at least three, at least four, and so forth. This may be achieved by the incorporation of a multivalent adaptor molecule, for example, a streptavidin molecule capable of simultaneous binding to multiple biotin-containing biotin molecules.

The invention also relates to use of this enzyme detection device for detecting enzyme cleavage activity in a sample and methods of detecting enzyme cleavage activity which rely upon this device.

The various aspects of the invention may rely upon a solid support in specific embodiments. Any suitable solid support is intended to be encompassed. The solid support may take the form of a bead (e.g. a sepharose or agarose bead) or a well (e.g. in a microplate) for example. In some embodiments, the solid support may form or define a liquid flow path for the test sample. In specific embodiments the solid support takes the form of a chromatographic medium.

In the case of kits of the invention, the solid support may be provided without the relevant capture molecules and/or binding molecules attached. The kits may also include instructions for use. Otherwise the kits comprise the same essential features as the devices of the invention. In those embodiments, the user of the kit may immobilize the capture molecules on the solid support to form the capture zone prior to use of the device with a test sample. The kit may, therefore, also comprise means for immobilizing the capture molecules on the solid support, and binding molecules where appropriate. The immobilizing means may comprise any suitable reagents to permit the capture zone to be formed. The solid support may be pre-formed with suitable immobilizing means. For example, the solid support may comprise biotin molecules arranged to interact with avidin (e.g. streptavidin) molecules that form (part of) the capture molecules. Of course, other binding pair interactions may be used to immobilize the capture molecules on the solid support to form a capture zone, as discussed herein and as would be readily understood by one skilled in the art.

Wherein the device is a flow device comprising a chromatographic medium as solid support, the capture molecules may be immobilized by directly binding to the medium or immobilized indirectly via binding to a carrier molecule, such as a protein, associated with, or bound to, the medium.

In embodiments where the solid support forms a liquid flow path for the test sample, the solid support may further comprise a sample application zone to which the sample is applied. The sample application zone may be pre-loaded with the indicator molecule, such that when the test sample is applied any enzyme in the sample acts upon at least one of the multiple cleavage sites of the indicator molecule within the sample application zone. The sample application zone may contain a barrier, which holds the sample in the sample application zone for a pre-determined period of time. This permits the sample to interact with the indicator molecule for a sufficient period to achieve measurable levels of cleavage. This may be 1, 2, 3, 4, 5, 6, 7, 8, 9, 10, 15, 20, 30, 60 minutes or more depending upon the enzyme to be detected, as would be readily understood by one skilled in the art. The barrier may be degraded by the sample, or otherwise removed, after this period of time thus allowing the sample to continue to flow through the device. Alternatively, the test sample and indicator molecule may be pre-mixed or pre-incubated prior to adding the mixture to the device, such as to the sample application zone. However, where the test sample and indicator molecule may be pre-mixed or pre-incubated it is possible to omit the sample application zone. Here, it may be possible to add the mixture directly to the capture zone to permit immobilization of the indicator molecules through interaction with the capture molecules. In some embodiments, the test sample may be applied to the chromatographic medium at a site upstream from the capture zone such that it is drawn, for example by capillary action, through the capture zone. The chromatographic medium may be made from any material through which a fluid is capable of passing, such as a fluidic channel or porous membrane. In certain embodiments of the invention, the chromatographic medium comprises a strip or membrane, for example a nitrocellulose strip or membrane.

Where binding molecules are employed (to define a detection zone) they are provided in the device in a manner that permits interaction with the part of fragment of the indicator molecule released as a consequence of cleavage at the at least one of the multiple cleavage sites. The binding molecules can be immobilized on the solid support in the same manner as the capture molecules (but in discrete regions to form the capture and detection zones respectively).

Depending upon the particular enzyme cleavage activity that is being detected, it may be necessary to incorporate suitable enzyme inhibitors into the devices or methods. This may be important to prevent the enzyme from acting upon other components of the device or method, such as the binding molecules or capture molecules. Where the test sample is pre-incubated with the indicator molecule, it may be advantageous to add an inhibitor of the enzyme activity at the end of the incubation period. This is preferably before the capture molecules and/or binding molecules (depending upon the embodiment concerned) come into contact with the test sample. Alternatively, the enzyme activity inhibitor or inhibitors may be included in the device at any point upstream of the capture molecules and/or binding molecules. This is upstream of the capture zone (per the discussion herein above). The inhibitor may be simply dried or passively adsorbed onto the device such that the test sample mobilises the inhibitor as it passes through the device. It should be noted that use of an inhibitor is not essential.

For example, some of the enzyme activities detected according to the invention such as specific protease activity may be sufficiently specific that the protease will not act on any other components of the device or method than the enzyme modifiable region of the indicator molecule. The cleavage sites of particular enzymes are well known in the art and can be used to design the various components of the devices and methods. For example, in silico screening may be performed (e.g. using freely available tools such as protein Basic Local Alignment Search Tool (protein BLAST) according to standard settings) to confirm that the cleavage site of the enzyme to be detected is not contained within any of the relevant molecules; such as the binding molecules and capture molecules. It is also possible to check for cross-reactivity by incubating the relevant molecules (e.g. binding molecules and capture molecules) with the enzyme activity to be tested and detecting whether cleavage occurs.

The solid support may further comprise a control zone, downstream of the capture zone in relation to sample flow, and the sample application zone if present, containing further binding molecules that may bind to a further molecule added to the sample or to the device and which flows with the sample through the device. The further molecule may be labelled, either directly or indirectly, with a reporter molecule. The control zone is spatially separated from the capture zone and detection zone (as appropriate), for example to produce two separate test lines if the reporter is bound or immobilized in each respective zone. This control zone is used to confirm that the test sample has passed through the entire device and confirms that the device is operating correctly. A positive signal is expected at the control zone independent of whether enzyme cleavage activity is present in the sample or not. The further binding molecules are selected based upon the nature of the further molecule added to the sample. The further molecules and further binding molecules may form a binding pair as defined herein. For example, if the further molecule is an antibody from a given species, e.g. a chicken or a goat, the further binding molecule may be an appropriate anti-species antibody. This permits immobilization of the binding molecule or further molecule at the control zone by virtue of a specific interaction. The further binding molecules may be immobilized in the control zone by any suitable means, for example by a covalent or non-covalent interaction.

### BRIEF DESCRIPTION OF THE DRAWINGS

The invention will now be described by way of example with respect to the accompanying drawings in which:

FIG. 1 is a schematic representation of an enzyme detection device according to one aspect of the invention. FIG. 1A shows the device in operation in the absence of protease in the sample. FIG. 1B shows the device in operation in the presence of protease in the sample.

FIG. 2 is a schematic representation of a further enzyme detection device according to one aspect of the invention. FIG. 2A shows the device in operation in the absence of protease in the sample. FIG. 2B shows the device in operation in the presence of protease in the sample.

FIG. 3 is a graph showing the improved sensitivity of detection of MMP activity achieved using a peptide containing two cleavage sites compared to an otherwise identical peptide substrate including a single cleavage site.

### DESCRIPTION OF PREFERRED EMBODIMENTS

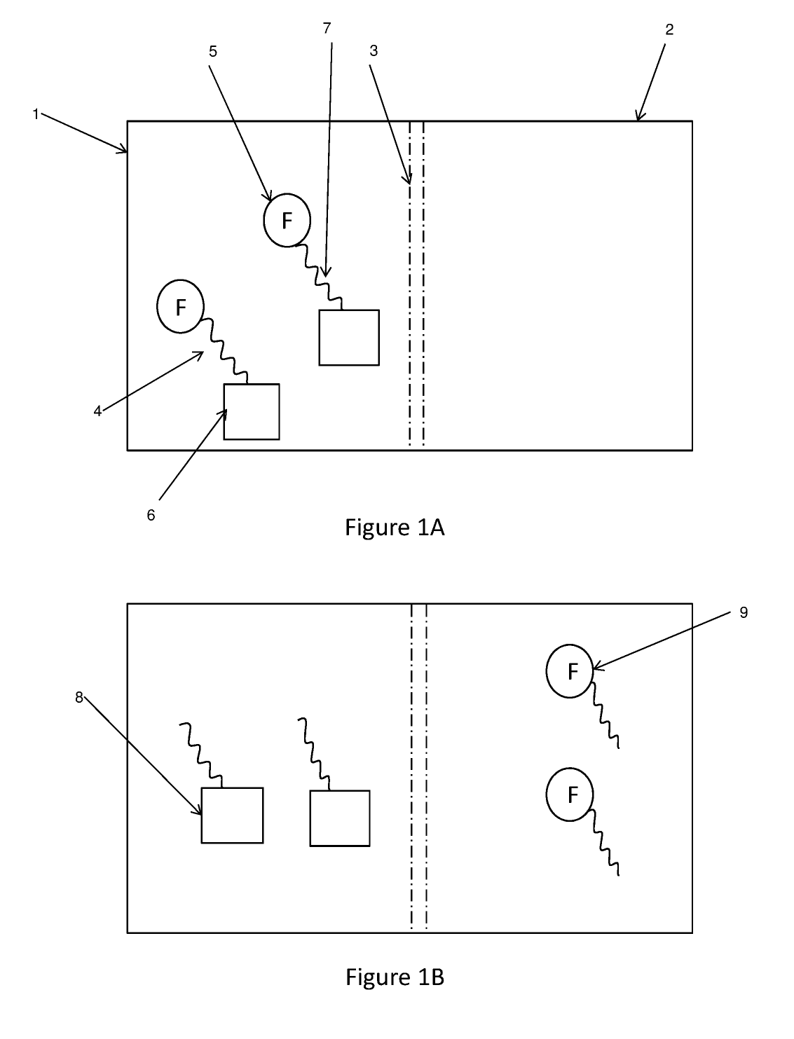

Referring to FIG. 1, an enzyme detection device comprises a reaction zone (1) and a detection zone (2). The zones are separated by a semi-permeable membrane (3). Initially contained in the reaction zone (2) is an indicator molecule (4). The indicator molecule (4) comprises a fluorophore (5) and an enzyme modifiable region comprising multiple separate cleavage sites (7). The indicator molecule is immobilized on a solid support in the form of a bead (6). The indicator molecule is too large to pass through the membrane (3). The solid support is a convenient but optional manner in which to ensure that the indicator molecule is above the size threshold of the membrane (3). Alternatively, the indicator molecule may simply comprise a further protein or peptide domain and may be a protein-based indicator molecule. The indicator molecule may be attached to a carrier protein such as albumin (e.g. BSA) in other embodiments.

In use, the test sample and indicator molecule (4) are added to the reaction zone (1). They may be pre-incubated together if desired prior to adding to the reaction zone (1). As shown in FIG. 1A, in the absence of protease activity in the sample, none of the multiple cleavage sites (7) is cleaved. Accordingly, the indicator molecule remains intact and the fluorophore (5) is unable to pass through the membrane (3) into the detection zone (2).

As shown in FIG. 1B, in the presence of protease activity in the sample, at least one of the multiple cleavage sites (7) is cleaved. Accordingly, the indicator molecule separates into a portion (8) that is optionally retained in the reaction zone because its size is larger than the threshold size of the membrane (3). The portion (8) may pass through the membrane (3) in other embodiments following cleavage as in this embodiment it does not affect the signal in the detection zone (2). Cleavage also produces a fragment of the indicator molecule containing the fluorophore (9). This fragment passes through the membrane (3) into the detection zone (2) where it is detected to show presence of protease in the sample.

In some embodiments the solid support (6) may be replaced by a fluorescence acceptor/quencher. In those embodiments, FRET between the fluorophore donor (5) and acceptor/quencher (6) prevents a signal being produced in the reaction zone. Cleavage releases the fluorophore containing fragment (9) which can pass through the membrane (3) and is detected in the detection zone (2). Preferably the portion of the indicator molecule containing the acceptor/quencher (8) after cleavage is unable to pass through the membrane (3), although this is not essential.

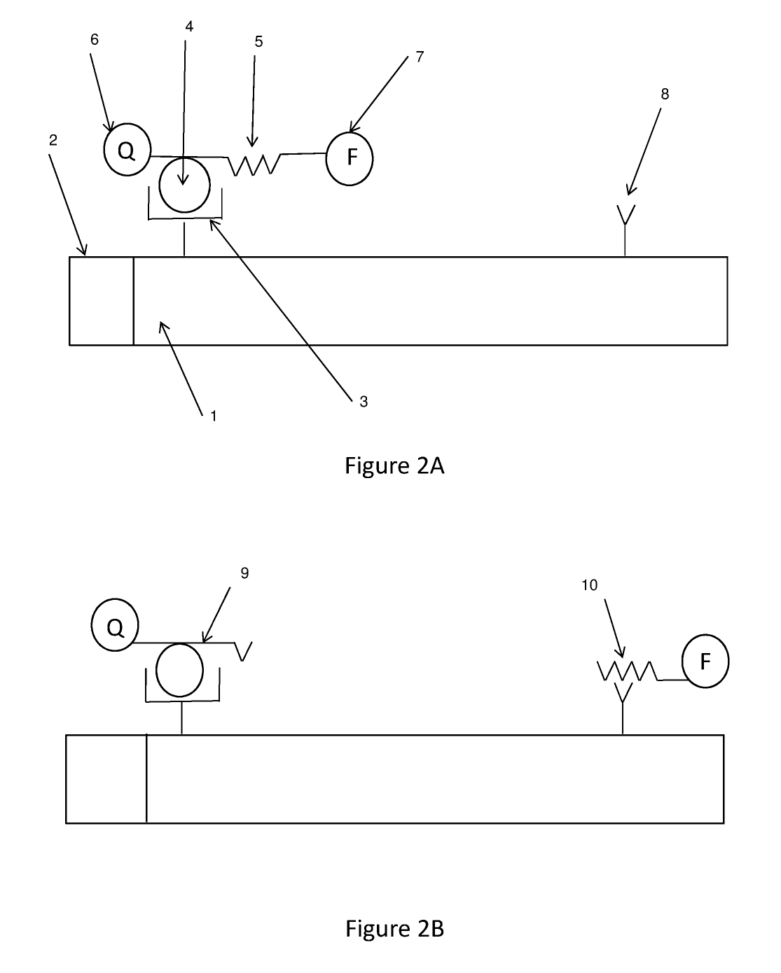

Referring now to FIG. 2, an enzyme detection device comprises a solid support (1) which provides a liquid flow path for the test sample (from left to right in the figure). The solid support optionally comprises a sample application zone (2) to which the sample is applied and then flows along the liquid flow path. Otherwise, the sample can be added directly to the capture molecules (3) which define a capture zone on the liquid flow path. The indicator molecule includes a capture site (4) which interacts with the capture molecules (3) to effectively immobilize the indicator molecules on the liquid flow path. The indicator molecules are preferably pre-incubated with the test sample prior to application to the device. In alternative embodiments, the indicator molecules and test samples are added to the optional sample application zone (2) approximately simultaneously. The captured indicator molecule comprises a fluorescence donor (7) and acceptor/quencher (6) pair. The donor (7) and acceptor (6) are separated by the enzyme modifiable region of the indicator molecule which contains multiple separate cleavage sites (5). Downstream of the capture zone are binding molecules (8) immobilized in the liquid flow path and which define a detection zone.

In the absence of protease activity in the sample, the entire indicator molecule remains immobilized at the capture zone. Due to the presence of FRET between the donor (7) and acceptor/quencher (6) fluorophores fluorescence emission by the donor is inhibited.

In the presence of protease activity in the sample, at least one of the multiple cleavage sites is cleaved. This produces a fragment of the indicator molecule containing the fluorophore (10) which is released and can be immobilized at the detection zone via interaction with the binding molecules (8). Fluorescence emission of the donor (7) can then be measured as an indicator of protease activity in the sample (increased relative to pre-cleavage levels due to the removal of FRET with the donor/quencher). The quencher remains attached to the indicator molecule in the portion immobilized at the capture zone via interaction between the capture molecules (3) and capture site (4).

The detection zone is optional and cleavage can simply be monitored by release of the fluorophore following cleavage. Similarly, immobilization of the indicator molecule can be in the reverse orientation such the cleavage results in the quencher as the “leaving fragment” (10) of the indicator molecule. In those embodiments, the fluorescence is measured at the capture zone because the fluorophore donor remains immobilized in the capture zone as it remains connected to the capture site (4) of the indicator molecules. Here, removal of quenching is measured as an increase in fluorescence intensity (at the emission wavelength) of the donor fluorophore.

The invention will be further understood with reference to the following experimental examples.

### EXAMPLES

### Example 1

### Flurogenic Peptide with Multiple Cleavable Sequences

2 flurogenic peptides with 1 (PCL0174 A4) or 2 (PCL0174 B1) cleavable sequences respectively were investigated with sensitivity towards Matrix-Metalloproteinase 9 (MMP9).

The sequences of the two peptides are:

PCL0174 A4 (1CS)=Lys(Mc Coumarin)-[GPQGIFGQ]-Lys(Dnp)G

PCL0174 B1 (2CS)=Lys(Mc Coumarin)-[GPQGIFGQ])-[GPQGIFGQ]-Lys(Dnp)G

Materials/Reagents

96-well assay plate, black polystyrene, Costar®, Corning Inc. (Cat. #3925, lot #25011023)

MMP buffer, Aq. Solution of 50 mM Tris, 100 mM sodium chloride, 10 mM Calcium Chloride, 50 μM 20 mM zinc chloride, 0.025% Brij 35, 0.05% sodium azide at pH 8.0.

Peptides PCL0174 A4 and PCL0174 B1 at a molar concentration of 8 mM

Testing Method
Peptides at a stock of 8 mM were diluted to 0.2 mM in MMP buffer
   2 μl+78 μl MMP buffer
5 μl sample (active MMP9 at 2 μg/ml)+190 μl MMP buffer added to well
5 μl of 0.2 mM peptide added per well
Read on a Fluorometer using the following settings:
   Excitation wavelength: 328 nm, bandwidth 5 nm
   Emission wavelength: 400 nm, bandwidth 5 nm
   Kinetic scan: 30 min, interval 1 min

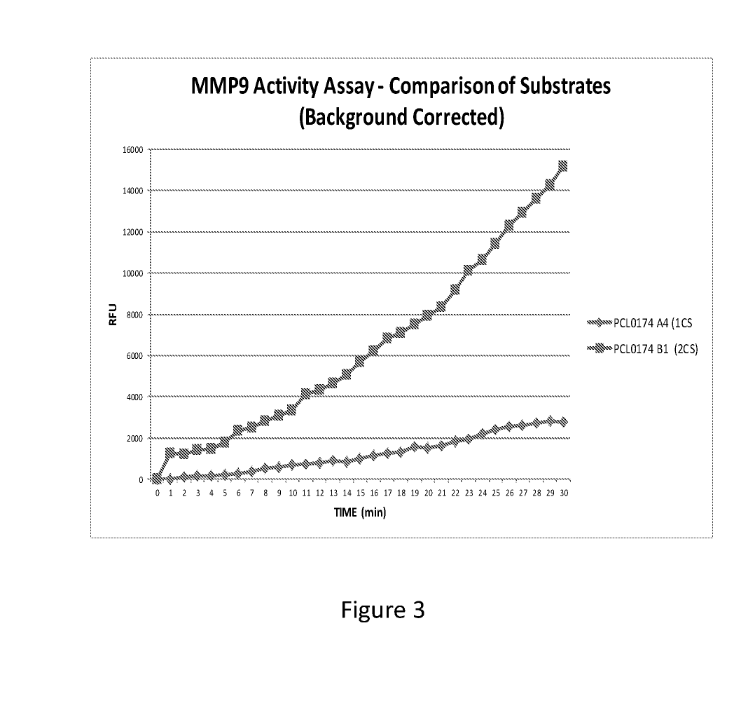

FIG. 3 demonstrates the sensitivity of the assay over 30 minutes with the 2 peptides, the same result (signal) can be achieved with the peptide with 2 CS at 8 minutes as the peptide with 1 CS at the running time of 30 minutes indicating that the sensitivity towards MMP9 can be improved with the addition of a further cleavable sequence.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate. Various publications are cited herein, the disclosures of which are incorporated by reference in their entireties.

1. An indicator molecule for use in the detection of enzyme cleavage activity in a test sample comprising:
(i) an enzyme modifiable region comprising multiple separate cleavage sites; and
(ii) a fluorophore,
wherein cleavage at one or more of the multiple separate cleavage sites causes a measurable change in fluorescence of the fluorophore.

2. The indicator of claim 1 wherein the measurable change in fluorescence is intensity, polarization or lifetime.

3. The indicator molecule of claim 1 further comprising a second fluorophore to create a donor and acceptor transfer pair.

4. The indicator molecule of claim 3 wherein the measurable change in fluorescence intensity is caused by the separation of the donor and acceptor transfer pair upon cleavage at any one of the multiple separate cleavage sites.

5. The indicator molecule of claim 3 wherein the acceptor is a quencher.

6. The indicator molecule of claim 5 wherein the fluorescence intensity of the donor is increased upon cleavage at any one of the multiple separate cleavage sites.

7. The indicator molecule of claim 1 comprising between 2 and 25 cleavage sites.

8-18. (canceled)

19. A method of detecting enzyme cleavage activity in a test sample, the method comprising:
a. bringing an indicator molecule into contact with the test sample, said indicator molecule comprising
i. an enzyme modifiable region comprising multiple separate cleavage sites; and
ii. a fluorophore,
wherein cleavage at one or more of the multiple separate cleavage sites causes a measurable change in fluorescence of the fluorophore.
b. detecting cleavage of at least one of the multiple separate cleavage sites by measuring a change in fluorescence of the fluorophore.

20. The method according to claim 19 which is performed in solution or which is a homogenous assay.

21. The method according to claim 19 wherein a change in fluorescence intensity, polarization or lifetime is measured.

22. The method according to claim 19 wherein the indicator molecule further comprises a second fluorophore to create a donor and acceptor transfer pair.

23. The method of claim 22 wherein the measured change in fluorescence is caused by the separation of the donor and acceptor transfer pair upon cleavage at any one of the multiple separate cleavage sites.

24. The method of claim 22 wherein the acceptor is a quencher, such that prior to cleavage fluorescence of the donor is quenched due to fluorescence energy transfer from the donor to the quencher.

25. The method of claim 24 wherein the fluorescence intensity of the donor is increased upon cleavage at any one of the multiple separate cleavage sites.

26. The method of claim 25 wherein the indicator molecule comprises between 2 and 25 cleavage sites.

27-41. (canceled)

42. An enzyme detection device for detecting enzyme cleavage activity in a test sample comprising:
a. an indicator molecule as claimed in claim 1; and
b. a housing comprising:
i. a reaction zone to which the indicator molecule and test sample is added
ii. a detection zone where fluorescence of the fluorophore is detected; and
iii. a membrane separating the reaction zone and detection zone, wherein the membrane is permeable to the fluorophore, or fragment of the indicator molecule containing the fluorophore, which is produced upon cleavage of the indicator molecule, but which is not permeable to the indicator molecule prior to cleavage.

43. The enzyme detection device of claim 42 further comprising:
c. A solid support upon which the indicator molecule may be, or is, immobilized.

44. The enzyme detection device of claim 42 wherein the indicator molecule further comprises a second fluorophore to create a donor and acceptor transfer pair and wherein the membrane is permeable to the donor fluorophore, or fragment of the indicator molecule containing the donor fluorophore which is produced upon cleavage of the indicator molecule, but which is not permeable to the indicator molecule prior to cleavage or the fragment of the indicator molecule containing the acceptor fluorophore which is produced upon cleavage of the indicator molecule.

45-46. (canceled)

47. An enzyme detection device for detecting the presence in a test sample of cleavage activity of an enzyme capable of cleaving a substrate, the device comprising:
a. an indicator molecule according to claim 1, wherein the indicator molecule contains a separate capture site which can be bound by a capture molecule irrespective of the state of modification of the enzyme modifiable region
b. a solid support comprising a capture zone to receive the test sample, wherein the capture zone comprises capture molecules capable of binding to the capture site of the indicator molecule.

48-53. (canceled)


SEQUENCE LISTING (1 sequence)

SEQ ID NO: 1 - protein, 8 aa, Artificial sequence
  Synthetic peptide
GPQGIFGQ